(19) United States
(12) Patent Application Publication
(10) Pub. No.: US 2021/0060322 A1
(43) Pub. Date: Mar. 4, 2021

(54) WEARABLE DEVICE WITH MICRONEEDLE ARRAY DELIVERY SYSTEM

(71) Applicant: EVONIK CORPORATION, PARSIPPANY, NJ (US)

(72) Inventor: Kevin Burton, Hoover, AL (US)

(21) Appl. No.: 16/960,875

(22) PCT Filed: Jan. 4, 2019
(86) PCT No.: PCT/US2019/012267

§ 371 Date: Jul. 8, 2020

Related U.S. Application Data
(60) Provisional application No. 62/615,245, filed on Jan. 9, 2018.

(51) Int. Cl. A61M 37/00; A61B 5/15; A61B 5/157

(52) U.S. Cl. CPC A61M 37/0015; A61B 5/150022; A61B 5/150984; A61M 2230/201; A61M 2037/0023; A61M 2037/0061; A61M 2037/003; A61B 5/157

(57) ABSTRACT

Described herein are wearable devices comprising a base coupled to a user, and a microneedle array supported by a base.

### BACKGROUND OF THE INVENTION

Human skin has three layers: the epidermis, the outermost layer of skin, which provides a waterproof barrier and creates our skin tone; the dermis, which is beneath the epidermis, and contains tough connective tissue, hair follicles, and sweat glands; and the hypodermis, which is a deeper subcutaneous tissue that is made of fat and connective tissue. An outermost layer of the epidermis is the stratum corneum, which functions to form a barrier to protect underlying tissues from infection, dehydration, chemicals and mechanical stresses.

Microneedle arrays are minimally invasive devices that are applied to the skin surface to deliver medicinal formulations through the skin. See, for example, FIG. 1, illustrating the layers of skin and a microneedle array coupled to the skin surface. Microneedles are typically 50-900 mm in height. Microneedles can be arranged in an array of up to 2000 cm² in various geometries and materials (e.g., silicon, metal, polymer) using microfabrication techniques. Microneedles are applied to the skin surface and painlessly pierce the epidermis, creating microscopic channels through which the medicinal formulations diffuse through the epidermis to the dermal microcirculation. Microneedles are long enough to penetrate to the dermis, but are short and narrow enough to avoid stimulation of dermal nerves or puncture of dermal blood vessels.

### SUMMARY OF THE INVENTION

In one embodiment, the invention provides a wearable device comprising a base coupled to a user, a recessed portion formed in the base, a microneedle array supported by the base wherein the microneedle array includes a plurality of microneedles, and an actuator coupled to the microneedle array to move the microneedle array into and out of the recessed portion in the base.

In another embodiment, the invention provides a wearable device comprising a base coupled to a user, a housing connected to the base, a microneedle array supported by the housing wherein the microneedle array includes a plurality of microneedles, and an actuator coupled to a shield in the housing to move the shield and expose the microneedle array to puncture the user's skin.

In yet another embodiment, the invention provides a method of analyzing a health status of a user. The method comprises activating a microneedle array in a wearable device to puncture the user's skin, the microneedle array including a plurality of hollow microneedles, collecting a fluid sample from the user via the hollow microneedles, analyzing the fluid sample to generate data of the user, transmitting the data to a remote electronic processor for review and diagnosis, and delivering a medicinal formulation from a reservoir in the wearable device to the user via the hollow microneedles based on the diagnosis.

Other aspects of the invention will become apparent by consideration of the detailed description and accompanying drawings.

### BRIEF DESCRIPTION OF THE DRAWINGS

FIG. 1 illustrates a cross-sectional view of human skin with a microneedle array applied to the skin.

FIG. 2A schematically illustrates application of a solid microneedle array to human skin.

FIG. 2B schematically illustrates application of a coated microneedle array to human skin.

FIG. 2C schematically illustrates application of a dissolvable/biodegradable microneedle array to human skin.

FIG. 2D schematically illustrates application of a hollow microneedle array to human skin.

FIG. 3 illustrates a microneedle array.

FIG. 4A illustrates an anchoring system for microneedles in a microneedle array according to an embodiment of the present invention.

FIG. 4B illustrates an anchoring system for microneedles in a microneedle array according to an embodiment of the present invention.

FIG. 4C illustrates an anchoring system for microneedles in a microneedle array according to an embodiment of the present invention.

FIG. 4D illustrates an anchoring system for microneedles in a microneedle array according to an embodiment of the present invention.

FIG. 4E illustrates an anchoring system for microneedles in a microneedle array according to an embodiment of the present invention.

FIG. 4F illustrates an anchoring system for microneedles in a microneedle array according to an embodiment of the present invention.

FIG. 4G illustrates an anchoring system for microneedles in a microneedle array according to an embodiment of the present invention.

FIG. 5A illustrates an anchoring system for microneedles in a microneedle array having a swellable portion according to an embodiment of the present invention.

FIG. 5B illustrates the anchoring system of FIG. 5A in an activated state.

FIG. 6A illustrates an anchoring system for microneedles in a microneedle array having a swellable portion according to an embodiment of the present invention.

FIG. 6B illustrates an anchoring system of FIG. 6A in an activated state.

FIG. 7 illustrates a portion of a wearable device with movable microneedle array according to an embodiment.

FIG. 8 illustrates a portion of a wearable device with movable microneedle array according to an embodiment.

FIG. 9 illustrates a portion of a wearable device with movable shield according to an embodiment.

FIG. 10 illustrates a block diagram of a wearable device with microneedle array according to an embodiment of the present invention.

FIG. 11 illustrates a flow chart of an example method for analyzing and delivering medicinal formulation to a user of the wearable device shown in FIG. 10.

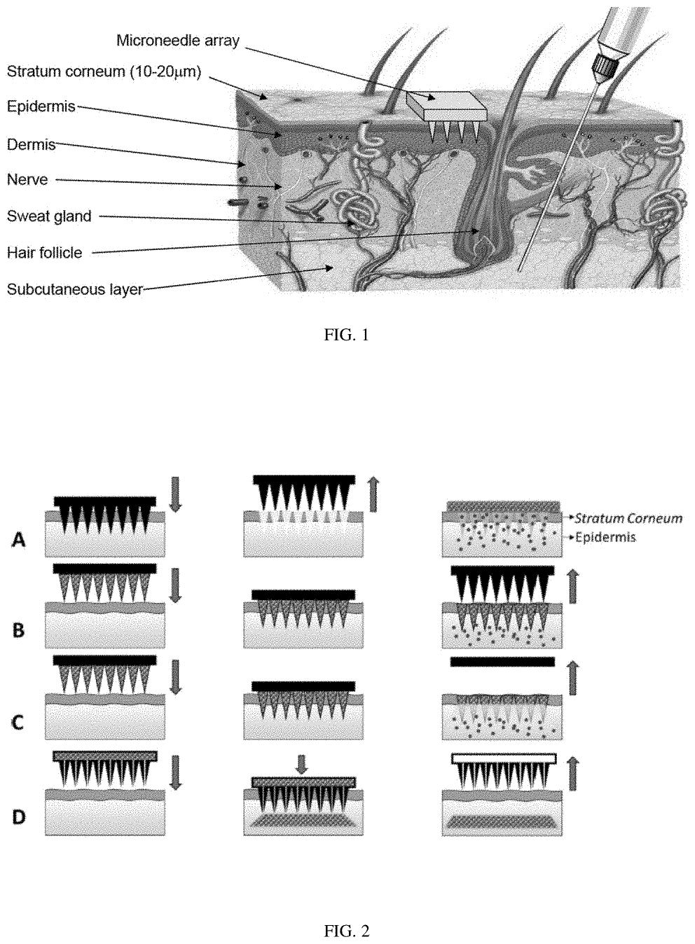

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

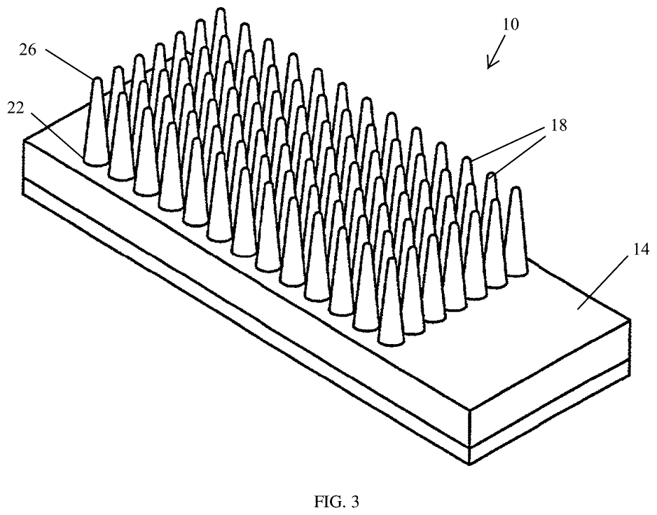

Microneedles can be solid, hollow, or dissolvable/biodegradable. With reference to FIG. 2A, a solid microneedle includes a smooth or seamless outer surface and typically comprises comprises a metal, a polymer, ceramic, semiconductor, material, organic, polymer, composite, silicon, silicon dioxide, chitosan, collagen, gelatin, maltose, dextrose, galactose, alginate, agarose, cellulose (such as carboxymethylcellulose or hydroxypropylcellulose), starch, hyaluronic acid, and combinations thereof. Examples of metals include, but are not limited to, dissolvable metals, pharmaceutical grade stainless steel, gold, titanium, nickel, iron, gold, tin, chromium, copper, alloys thereof, and combinations thereof. Examples of polymers may include, but are not limited to, glycolic acid polylactide, polyglycolide, poly lactide, polylactide-co-glycolide, and copolymers with polyethylene glycol (PEG), polyanhydrides, poly(ortho)esters, polyurethanes, poly(butyric acid), poly(valeric acid), poly(lactide-co-caprolactone), and combinations thereof. Examples of non-biodegradable polymers include, but are not limited to, polycarbonate, polymethacrylic acid, ethylenevinyl acetate, polytetrafluorethylene, polyesters, polymers of hydroxy acids such as lactic acid, and combinations thereof. A solid microneedle punctures skin to create temporary microchannels in the epidermis. The solid microneedle is removed from the skin before application of a medicinal formulation (e.g., a patch having a medicinal formulation embedded therein, solution, cream, gel, or other applicator). The medicinal formulation permeates through the microchannels by passive diffusion.

Solid microneedles may be coated with a medicinal formulation prior to insertion into the skin. Coated microneedles (FIG. 2B) are typically prepared by coating a medicinal formulation onto the microneedle outer surface prior to application to the skin. Coated microneedles are employed for the rapid cutaneous delivery of therapeutic agents including small molecules and macromolecules, such as vaccines, proteins, peptides, and DNA to the skin.

A dissolvable/biodegradable microneedle shown in FIG. 2C comprises a material that dissolves or biodegrades while embedded in the skin. A dissolvable/biodegradable microneedle releases its medicinal formulation as the material dissolves or biodegrades in the skin. Dissolvable/biodegradable microneedles are fabricated by micro-moulding soluble matrices, generally a biocompatible polymer or sugar, including the active substance. After insertion of the microneedle into skin, the tip begins to dissolve upon contact with skin interstitial fluid. The medicinal formulation is then released over time. The release kinetics of the medicinal formulation depends upon the constituent polymers' dissolution rate. Therefore, controlled medicinal formulation delivery is achievable by adjusting the polymeric composition of the microneedle, or by modification of the microneedle fabrication process. Dissolvable/biodegradable microneedles provide several advantages. One benefit is the low cost of polymeric materials and their relatively facile fabrication by micromoulding processes at ambient temperatures, which typically allow for straightforward industrial mass production. Various materials, including poly(vinyl alcohol) (PVA), poly(vinylpyrrolidone) (PVP), dextran, carboxymethyl cellulose (CMC), chondroitin sulfate and sugars have all been used to produce this type of microneedle. The use of water-soluble materials eliminates the potential risk of leaving biohazardous sharp waste in the skin. Moreover, safe microneedle disposal is facilitated, since the microneedles are, by definition, self-disabling. One disadvantage of a dissolvable/biodegradable microneedle is the deposition of polymer in skin, possibly making these systems undesirable if they are likely to be used on an ongoing basis.

More specifically, a biodegradable microneedle is produced using biodegradable polymers, including , for example, poly(lactic acid), chitosan, poly(glycolic acid), or poly(lactide-co-glycolide) (PLGA), glycolic acid polylactide, polyglycolide, polylactide-co-glycolide, and copolymers with polyethylene glycol (PEG), polyanhydrides, poly(ortho)esters, polyurethanes, poly(butyric acid), poly(valeric acid), and poly(lactide-co-caprolactone), as well as any of the materials described above with respect to FIG. 2A, to form the matrix. After insertion into skin, the microneedles degrade, rather than dissolve in the skin, releasing their medicinal formulation. Release could possibly be sustained for months by choosing the appropriate polymer. Since biodegradation typically produces small molecules by hydrolysis, polymer is not deposited in the skin indefinitely. However, such microneedles typically require high temperatures during manufacture, which may damage biomolecular medicinal formulations.

With reference to FIG. 2D, a hollow microneedle includes a bore formed therein capable of storing a medicinal formulation. After puncture of the skin with a hollow microneedle, the medicinal formulation is released, diffused, or pressure or electrically-driven through the bore from a supply source of the medicinal formulation. A hollow microneedle allows continuous delivery of molecules across the skin through the bore. Hollow microneedles are made from a range of materials, including silicon and metal, glass, polymers, and ceramic. The use of hollow microneedles is limited due to the potential for clogging of the needle openings with tissue during insertion and the flow resistance, due to dense dermal tissue compressed around the tip during insertion.

In some constructions, a microneedle array may include microneedles that all have the same structure. For example, all of the microneedles in the array are solid or all of the microneedles are hollow. In other constructions, a microneedle array may include microneedles having different structures. For example, some of the microneedles in the array are solid and some of the microneedles are hollow. There can be a pattern to the array. For example, the outer microneedles are solid while the inner microneedles are hollow. As another example, a first row comprises solid microneedles, the next adjacent row comprises hollow microneedles, in a continuing alternating pattern. Additional patterns of arrangement of the microneedles in an array are also contemplated.

As noted above, a microneedle array can be utilized for short-term or long-term use depending on the application and the medicinal formulation. For a longer-term use, such as, for example, one week to 6 months, it would be desirable to provide an anchoring system on the microneedles so that the microneedles remain in position during the term of use.

As shown in FIG. 3, a microneedle array 10 includes a base plate 14 and a plurality of microneedles 18 extending from the base plate 14. The base plate 14 may be rigid or flexible depending on the materials used in or the application of the microneedle array 10. The microneedles 18 all extend from the base plate 14 in the same direction. The microneedles 18 each have a base 22 integrally formed with the base plate 14 and a tip 26. The base of the microneedle denotes a proximal end, and the tip denotes a distal end of the microneedle 18. By way of example, the microneedles 18 as illustrated in FIG. 3 have a conical shape where the flat base of the cone is integrally formed with the base plate 14 and tapers smoothly from the flat base to a tip or apex. In other constructions, the shape of the microneedles 18 may be cylindrical (with or without a tapered tip), pyramidal, and the like.

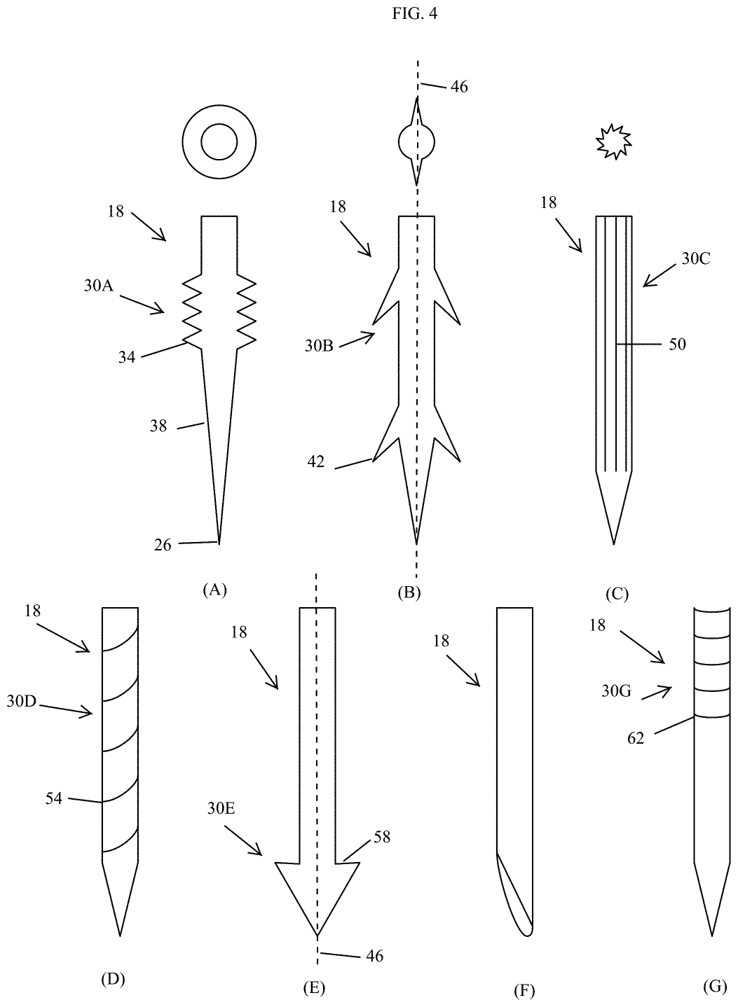

FIGS. 4A-G illustrate an anchoring system 30 integrated with the microneedles 18. FIGS. 4A-G illustrate a single microneedle 18 incorporating the particular anchoring system 30, however it is noted that all or just some of the microneedles 18 in the array 10 can include the particular anchoring system 30.

With reference to FIG. 4A, the anchoring system 30A includes a plurality of protrusions 34 extending radially and circumferentially from an outer surface 38 of the microneedle 18. The protrusions 34 extend perpendicularly (within about 1-2° of a right angle) from the outer surface 38. The protrusions 34 are adjacent to one another meaning there is no visible outer surface between the protrusions 34. The protrusions 34 are positioned in a middle portion of the microneedle 18 and closer to the base 22 than the tip 26. The anchoring system 30A is shown having four protrusions 34, however it is noted that more or fewer protrusions 34 are contemplated.

FIG. 4B illustrates a microneedle 18 having an anchoring system 30B. In this construction, a plurality of barbs 42 extend radially from the outer surface 38 of the microneedle 18. The barbs 42 are oriented at an angle relative to a longitudinal axis 46 of the microneedle 18. A tip of the barb 42 is oriented in the direction of the tip 26 of the microneedle 18. The barbs 42 are positioned at particular locations on the outer surface 38 of the microneedle 18. For example, a first set includes two barbs 42 that are positioned radially opposite one another and toward a distal end of the microneedle 18 while a second set, which includes two barbs are positioned radially opposite one another and toward a proximal end of the microneedle 18. As illustrated, the first set of barbs and the second set of barbs are aligned relative to one another when viewing the cross-sectional drawing, however in other constructions, the second set of barbs may be angularly offset from the first set of barbs.

FIG. 4C illustrates another embodiment of an anchoring system 30C. The anchoring system 30C includes a plurality of grooves 50 formed in the outer surface 38 of the microneedle 18. The grooves 50 are arranged circumferentially around the outer surface 38 with a consistent pattern. The grooves 50 extend between the base 22 and the tip 26 of the microneedle 18. In other constructions of this anchoring system 30C, some or all of the grooves 50 may extend partially along the length of the microneedle 18. In other constructions, the grooves 50 may extend circumferentially around the outer surface 38 in an irregular pattern.

With reference to FIG. 4D, the microneedle 18 shown therein includes an anchoring system 30D including a thread or a tapering groove 54 that spirals toward the tip 26 formed within the outer surface 38. The tapering groove 54, as illustrated extends from the proximal end to the distal end of the microneedle 18. It is noted that the tapering groove 54 may be formed in certain areas of the outer surface 38 and not fully extend from the proximal end to the distal end. It is also noted that the tapering groove 54 may be equally spaced (as illustrated) or may vary in certain areas of the outer surface 38.

FIG. 4E illustrates the microneedle 18 having an anchoring system 30E. The anchoring system 30E includes a radially inward step 58 near the tip 26 of the microneedle 18. The step 58 as illustrated is oriented perpendicularly (within about 1-2° of a right angle) with respect to the longitudinal axis 46, however it is possible that the step 58 may be angularly oriented with respect to the longitudinal axis 46 in other constructions.

FIG. 4F illustrates a further construction of an anchoring system 30F. The anchoring system 30F includes a bevel 60 defining an edge at the tip 26 of the microneedle 18 that assists in securing the microneedle 18 in position. In addition to the bevel 60, the microneedle 18 may include other anchoring systems described herein.

With reference to FIG. 4G, the microneedle 18 includes yet another possible anchoring system 30G. In this construction, the anchoring system 30G includes a plurality of circumferential grooves 62 formed in the outer surface 38 of the microneedle 18. The grooves 62 are positioned at the proximal end of the microneedle 18.

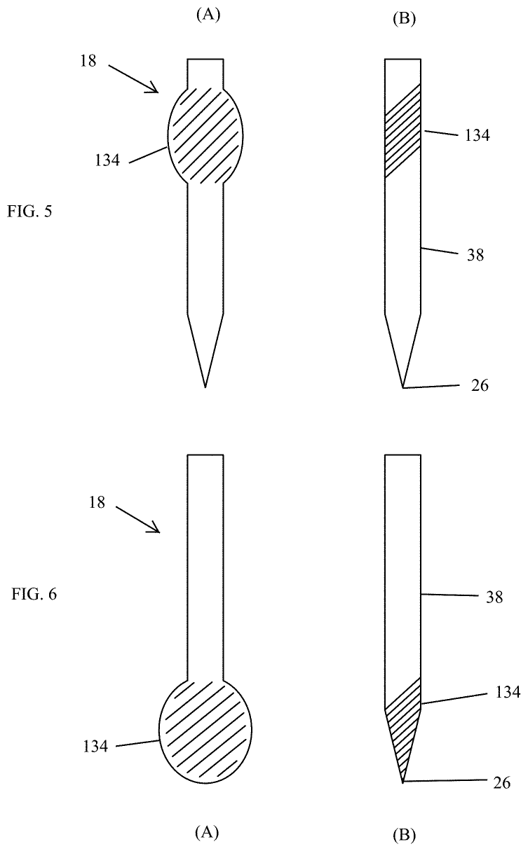

FIGS. 5A-B and FIGS. 6A-B illustrate an anchoring system 130 integrated with the microneedles 18. FIGS. 5A-B and FIGS. 6A-B illustrate a single microneedle 18 incorporating the particular anchoring system 130, however it is noted that all or just some of the microneedles 18 in the array 10 can include the particular anchoring system 130.

The microneedle 18 shown in FIGS. 5A-B and FIGS. 6A-B may be formed of the same materials set forth above with respect to FIG. 2A. In certain locations of the microneedle 18, a swellable portion 134 is formed in the polymer structure of the microneedle 18. The swellable portion 134 also comprises material such as polymethlmethacrylate, polyethylene glycol, hydrogels, superabsorbers, polyvinylpyrrolidone, polyvinyl alcohol, polyacrylic acid, dextran, polysaccharides, cellulosics (CMC, HPMC), hydroxypropyl methacrylamide, xanthan gum, pectins, collagen, gelatin, chitosan, alginate, hyaluronic acid, albumin, starch, polysiloxanylalkyl esters and combinations thereof. The swellable portion 134, in some constructions, may include a polymer that is different than the rest of the material forming the microneedle 18. The swellable portion 134 is capable of changing shape after it comes in contact with water from the skin or other fat from the body after implantation. The materials used for the swellable portion 134 may be selected based on their ability to change when exposed to external stimulus, such as body temperature, pH, light, magnetic field, or ultraviolet light.

With reference to FIGS. 5A-B, the swellable portion 134 is positioned closer to the proximal end than the distal end of the microneedle 18. In FIGS. 6A-B, the swellable portion 134 is positioned at the tip 26 or distal end of the microneedle 18. The swellable portion 134 is generally collinear with the outer surface 38 of the microneedle 18 prior to insertion (see FIGS. 5A and 6A) and then expands radially outward to achieve a greater circumference than the outer surface 38 after insertion (see FIGS. 5B and 6B) due to absorption of water, fat or other bodily fluids or other shape-changing property occurs.

The microneedles 18 in the microneedle array 10 may include a coating, such as a lubricious coating for ease of insertion. The coating can be applied to solid or hollow microneedles and to any of the microneedle constructions disclosed herein. The coating may extend the full length of the microneedles 18 or only partially along the length. For example, the coating may be applied at the tip of the microneedles and extend for a short distance along the shaft of the microneedles 18. By employing a lubricious coating on the outer surface 38 of the microneedles 18, the insertion is easier thereby leading to the ability to provide more microneedles 10 in the array 10. This would lead to an increased dosage amount of the medicinal formulation provided in the microneedle array 10. Suitable lubricious coatings include, but are not limited, to polyvinylpyrrolidone (PVP), polyurethanes, polyacrylic acid, polyethylene oxide, polysaccharides, hydrophobic polymers such as polytetrafluoroethylene and silicone. The lubricious coatings may have a suitable lubricity as measured by [INSERT], thickness [INSERT RANGES], surface friction as measured by [INSERT AND PROVIDE RANGES], contact angle as measured by [INSERT AND PROVIDE RANGES], and/or viscosity [INSERT RANGES].

In other constructions, the microneedles 18 may include a coating with antimicrobial agents to allow for long term use of the array 10. Suitable antimicrobial agents include, but are not limited to Penicillins, Penicillin V, Penicillin G, Amoxicillin, Ampicillin, Cloxacillin, Methicillin, Amoxicillin+Clavulanate (Augmentin), Ticarcillin+Clavulanate, Nafcillin, 1st Generation Cephalosporins, Cephalexin (Keflex), Cefazolin, Cefadroxil, (LEXie DROpped ZOLa), 2nd Generation Cephalosporins, Ceflaclor, Cefuroxime, (LACking URine), 3rd Generation Cephalosporins, Cefotaxime, Cefoperazone, Cephtriaxone, 4th Generation Cephalosporins, Cefepime, Tetracyclines, Tetracycline, Minocycline, Doxycycline, Macrolides, Azithromycin, Erithromycin, Clarithromycin, Lincosamides/Lincosamines, Clindamycin (Cleocin), Sulfonamides/Sulfa Drugs, Sulfamethoxazole-Trimethoprim (generic), (Bactrim), (Cotrim), (Septra), Fluoroquinolones, Ciprofloxacin (Cipro), Norfloxacin, Ofloxacin, Levofloxacin, Aminoglycosides, Streptomycin, Tobramycin, Gentamycin, Amikacin.

In fabricating the microneedle array 10, the coating may be applied by dip coating or spray coating onto the microneedles 18. Alternatively, in a molding process of the microneedle array 10, the coating could be added to the mold such that an outer layer of the microneedles 18 contain the coating upon completion of the fabrication process.

In conventional microneedle arrays with microneedles fabricated as dissolvable or biodegradable, the microneedles are comprised of a non-resorbable metal or a resorbable or non-resorbable polymer material. In contrast, embodiments of the invention include microneedles comprising a bioresorbable metal, such as magnesium, zinc, iron, tungsten, molybdenum, silver, gold, platinum, alloys thereof, other water soluble metals, heir alloy, and combinations thereof. Magnesium employed as the microneedle material would dissolve after application and release the medicinal formulation into the bod to elicit its biological response. A resorbable metal microneedle array offers an advantage of allowing a more structurally stable configuration that would enable the formation of a hollow resorbable needle. Suitable non-resorbable materials may have a suitable weight percentage of [INSERT] or biodegradability as measured by [INSERT AND PROVIDE RANGES].

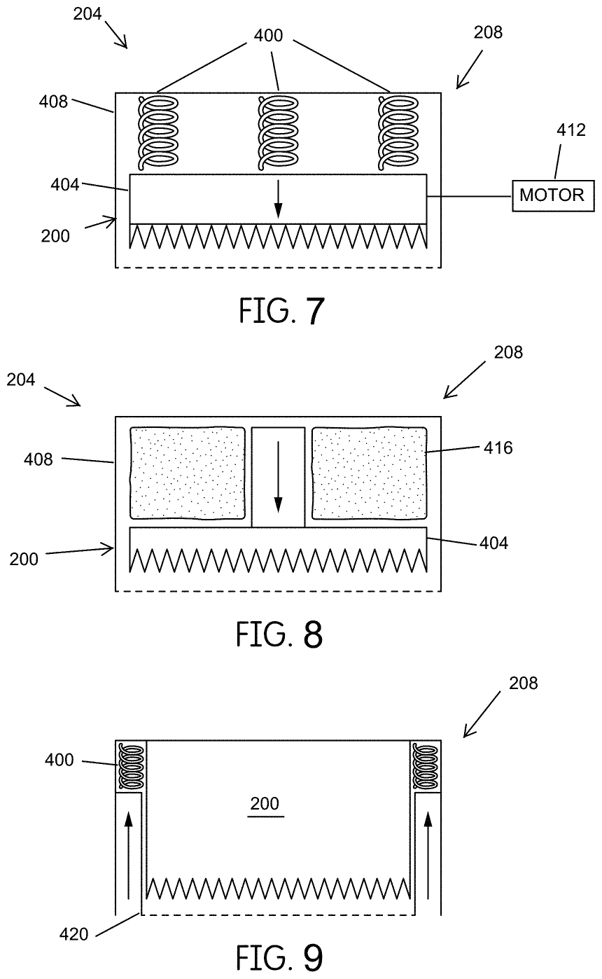

With reference to FIGS. 7-11, in some constructions a microneedle array 200 can be integrated with a wearable device 204, such as, a ring, a bracelet, a watch, or the like. The microneedle array 200 may comprise solid, hollow, coated, and/or dissolvable/biodegradable microneedles or a combination thereof as described above. Some or all of the microneedles in the array 200 can include an anchoring system and/or a coating as described above. Some or all of the microneedles in the array 200 also can comprise resorbable material as described above. The microneedle array 200 is disposable and can be replaced with a new microneedle array as needed.

The wearable device 204 can include an actuator 208 that interfaces with the microneedle array 200 to insert and retract the microneedle array 200 as needed or as programmed. The actuator 208 can move the microneedle array 200 toward the skin and apply a force until the microneedles puncture the skin. The actuator 208 also can retract the microneedle array 200 from the skin to an area (e.g., a recessed portion or cavity formed in the wearable device) within the wearable device 204 such that the microneedles are not in contact with the skin. For example, the microneedle array 200 can be actuated to sample the interstitial fluid on a periodic basis to determine blood glucose levels. In another example, the microneedle array 200 can be actuated to deliver a medicinal formulation to the user for a period of time until the microneedle array 200 is retracted.

As illustrated in FIG. 7, in one construction, the actuator 208 includes a spring 400 connected to a base plate 404 of the microneedle array 200 and a housing 408 on the wearable device 204. As illustrated, the actuator 208 is shown with three springs 400, however an actuator with more than or less than three springs 400 is also contemplated. The actuator 208 also includes a motor 412 coupled to the base plate 404. In operation, when the motor 412 receives a signal to move the microneedle array 200 to puncture the user's skin, the motor 412 moves the microneedle array 200 toward the user's skin. The springs 400 are stretched or lengthened. When the microneedle array 200 is to be retracted from the user's skin, the motor 412 is disconnected from the microneedle array 200 and the springs 400 pull or retract the microneedle array 200 from the skin and into the cavity.

With reference to FIG. 8, in another construction, the actuator 208 includes a solenoid 416. In operation, when the solenoid 416 receives a signal to move the microneedle array 200, a magnetic field is generated to thereby move the armature, which is coupled to the base plate 404 of the microneedle array 200. The electric current applied to the coil of the solenoid generates a magnetic field to thereby provide a force on the armature, which moves the microneedle array 200 to puncture the user's skin.

With reference to FIG. 9, in another construction, the actuator 208 is linked to a shield 420 instead of the microneedle array 200. The microneedle array 200 remains fixed in position. In this construction, the actuator 208 includes a spring 400 coupled to the shield 420. In operation, when the springs 400 receive a signal, the springs 400 contract or pull to move the shield 420 to expose the microneedle array 200. Upon retraction of the shield 420, the user's skin is punctured. When the microneedle array 200 is to be retracted from the user's skin, the motor 412 is disconnected from the microneedle array 200 and the springs 400 pull or retract the microneedle array 200 from the skin and into the cavity.

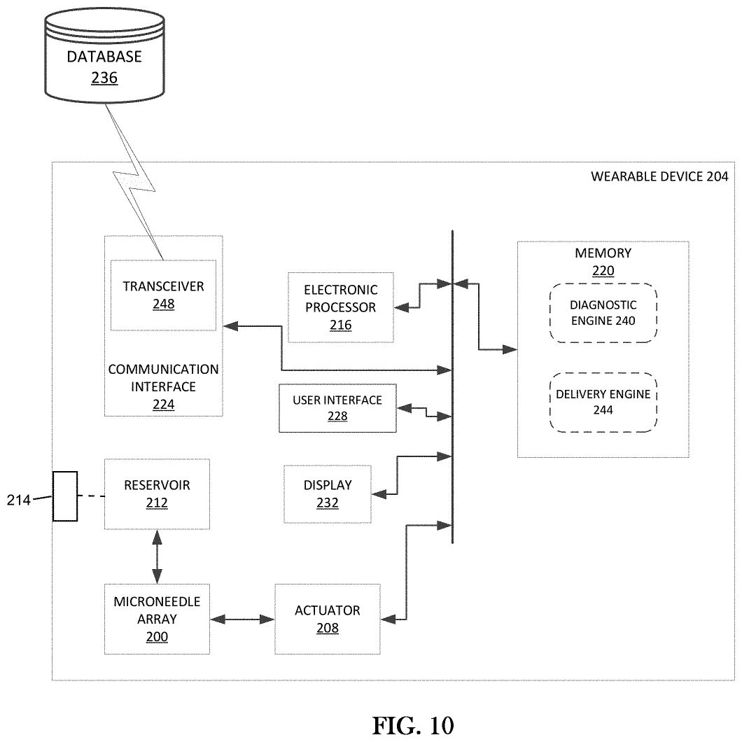

As illustrated in FIG. 10, the wearable device 204 can include a reservoir 212 for a medicinal formulation or therapeutic agent. Upon activation, the microneedle array 200 can deliver the medicinal formulation from the reservoir 212 to the skin. As such, the wearable device 204 operates as a monitoring device, diagnostic device (e.g., lab on a chip) or a combination monitoring, diagnostic, and drug delivery device operated through various feedback loops. The reservoir 212 may be embedded within the wearable device 204. In other constructions, the reservoir 212 may be external to the wearable device and removably connectable to a port 214 on the wearable device and/or array 200 such that the reservoir is replaceable or refillable. The port 214 can provide for multiple medicinal formulations to be delivered to the user by switching the reservoir 212 that is coupled to the port 214. In yet other constructions, the reservoir 212 may be integrated into the base of the microneedle array 200.

With continued reference to FIG. 10, the wearable device 204 includes an electronic processor 216, a memory 220, a communication interface 224, a user interface 228, and a display 232. The illustrated components, along with other various modules and components are coupled to each other by or through one or more control or data buses that enable communication therebetween. The use of control and data buses for the interconnection between and exchange of information among the various modules and components would be apparent to a person skilled in the art in view of the description provided herein. The wearable device 204 is presented as an example that may be programmed and configured to carry out the functions described herein. In some embodiments, components of the wearable device 204 may be separately implemented, and may be communicatively coupled by a bus or by a suitable communication network. It should be understood that, in other constructions, the wearable device 204 includes additional, fewer, or different components than those illustrated in FIG. 10.

As illustrated in FIG. 10, in some embodiments, the wearable device 204 is communicatively coupled to, and writes data to and from, one or more of a remote server or a database 236. The database 236 may be a database housed on a suitable database server communicatively coupled to and accessible by the wearable device 204. In alternative embodiments, the database 236 may be part of a cloud-based database system accessible by the wearable device 204 over one or more networks. In some embodiments, all or part of the database 236 may be locally stored on the wearable device 204, for example within the memory 220. In some embodiments, as described below, the database 236 electronically stores one or more user characteristics (e.g., personal data and history, medical data, and the like), medicinal formulation information, dosage information, threshold information, diagnosis information, historical data, dosing interval, amount of therapeutic delivered, pharmacological data, pharmacokinetic data, pharmacodynamic data, and environmental data. It should be understood that in some embodiments, the wearable device 204 may be configured to communicate and implement the methods described herein with more than one database.

The electronic processor 216 obtains and provides information (for example, from the memory 220 and/or the communication interface 224), and processes the information by executing one or more software instructions or modules, capable of being stored, for example, in a random access memory (“RAM”) area of the memory 220 or a read only memory (“ROM”) of the memory 220 or another non-transitory computer readable medium (not shown). The software can include firmware, one or more applications, program data, filters, rules, one or more program modules, and other executable instructions. The electronic processor 216 is configured to retrieve from the memory 220 and execute, among other things, software related to the control processes and methods described herein.

The memory 220 can include one or more non-transitory computer-readable media, and includes a program storage area and a data storage area. The program storage area and the data storage area can include combinations of different types of memory, as described herein. The memory 220 may take the form of any non-transitory computer-readable medium. In the embodiment illustrated, the memory 220 stores, among other things, a diagnostic engine 240 and a delivery engine 244. The diagnostic engine 240 transmits instructions to the actuator to actuate the microneedle array 200 (or the shield 420). After actuation of the microneedle array 200 (or shield 420) and puncture of the user, the diagnostic engine 240 functions to analyze bodily fluid samples (e.g., interstitial fluid) captured by the microneedles in the array 200 to detect and identify one or more characteristic details of the user, such as a disease state (e.g., diabetes, high cholesterol (hyperlipidemia), high blood pressure (hypertension), asthma, COPD, arthritis, Crohn's disease, heart disease, HIV/AIDS, lupus, mental diseases (e.g., schizophrenia, depression, mania), Alzheimer's, kidney disease, hyperthyroidism, haemophilia, glaucoma, dysrhythmia, and allergies) or whether certain biomarkers are present in the fluid sample. The analysis details can be stored in memory 220 and/or transmitted to the database 236 (for storage and/or comparison to a threshold or a standard).

Depending on needs and use, while the microneedle array 200 is positioned in the user's skin, the delivery engine 244 can receive data from the diagnostic engine 240 to activate the reservoir to deliver a medicinal formulation or therapeutic agent to the user as needed and based on the analysis performed by the diagnostic engine 240. For example, if the diagnostic engine 240 determines that the user's blood glucose level is low, the diagnostic engine 240 can deliver instructions to the delivery engine 244 to provide insulin to the user through the microneedle array 200. The delivery engine 244 can then provide instructions to the actuator 208 to retract the microneedle array 200 (or the shield 420) from the skin and store the microneedle array 200 until next activation. Alternatively, the array 200 may remain in position in the skin until a follow-up diagnostic check of a new bodily fluid sample.

The communication interface 224 may include a transceiver 248 for wirelessly coupling to wireless networks (for example, land mobile radio (LMR) networks, Long Term Evolution (LTE) networks, Global System for Mobile Communications (or Groupe Special Mobile (GSM)) networks, Code Division Multiple Access (CDMA) networks, Evolution-Data Optimized (EV-DO) networks, Enhanced Data Rates for GSM Evolution (EDGE) networks, 3G networks, 4G networks, combinations or derivatives thereof, and other suitable networks, including future-developed networks. Alternatively, or in addition, the communication interface 224 may include a connector or port for receiving a connection to a wired network (for example, Ethernet). The transceiver 248 obtains information and signals from, and provides information and signals to, (for example, over one or more wired and/or wireless connections) devices both internal and external to the wearable device 204. Although the transceiver 248 is illustrated as a single component, in some embodiments the transceiver 248 is implemented as a transmitter and receiver separate from each other.

The user interface 228 operates to receive input from, for example, a user of the wearable device 204, to provide system output, or a combination of both. The user interface 228 obtains information and signals from, and provides information and signals to, (for example, over one or more wired and/or wireless connections) devices both internal and external to the wearable device 204. Input may be provided via, for example, a keypad, a microphone, soft keys, icons, or soft buttons on the display 232, buttons, and the like. System output may be provided via the display 232. The display 232 is a suitable display such as, for example, a liquid crystal display (LCD) touch screen, or an organic light-emitting diode (OLED) touch screen. The wearable device 204 may implement a graphical user interface (GUI) (for example, generated by the electronic processor 216, from instructions and data stored in the memory 220, and presented on the display 232), that enables a user to interact with the wearable device 204.

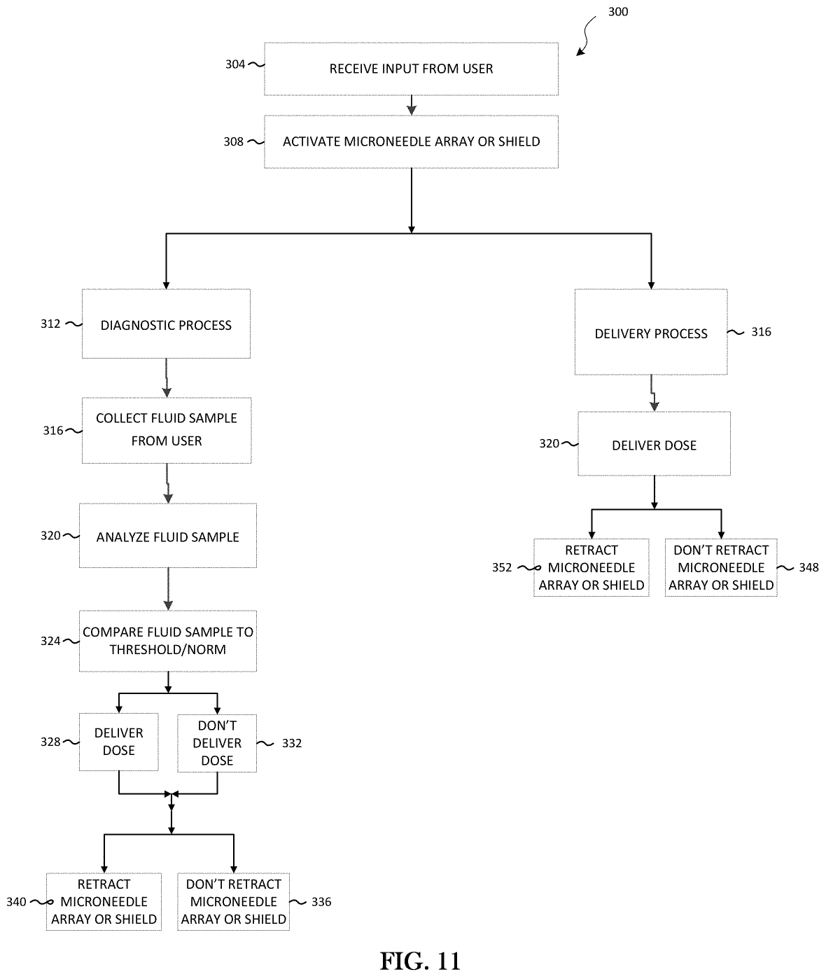

FIG. 11 illustrates an example method 300 for analyzing and delivering medicinal formulation to a user of the wearable device 204. The method 300 is described as being performed by the electronic processor 216 executing the diagnostic engine 240 and delivery engine 244. However, it should be understood that in some embodiments, portions of the method 300 may be performed by other devices, including for example, a computer located remotely from the wearable device 204 and wirelessly coupled to the wearable device 204.

At block 304, the electronic processor 216 receives input from the user via the user interface 228 (e.g., a touch screen on the wearable device 204). The input can include instructions selected by the user based on a questionnaire pre-programmed in the memory 220. The questionnaire may ask the user to select answers related to diagnosis or disease, timing for activation of the microneedle array 200, timing for delivery of medicinal formulation by the microneedle array 200 to the skin, and the like. It is noted that the instructions needed for operation of the wearable device 204 may have been added or pre-programmed by a caregiver or other medical personnel.

At block 308, at a predetermined time (as input from step 304), the electronic processor 216, using the delivery engine 244, provides an instruction to the actuator 208 to activate the microneedle array 200 (or the shield 420) to puncture the user's skin. From here, the wearable device 204 may perform a diagnostic process or a standard delivery process.

In the diagnostic process, at block 312, the microneedles (e.g., hollow) come into contact with and collect a fluid sample (block 316) from the user. The fluid sample is analyzed (block 320) by the diagnostic engine 240 for certain biomarkers, fluid concentration, and other information, such as, but not limited to pathogens, therapeutic drug levels, toxins, pH, gas levels, electrolytes, hemoglobin, glucose, LDL/HDL, triglyerides, and fibrogen. The data from the analyzed fluid sample can be compared (block 324) to threshold data or norms stored in the database 236 (either locally on the wearable device 204 or in a remote database) predetermined for the user or for general population. Based on the results of the comparison, the diagnostic engine 240 can communicate with the delivery engine 244 to either deliver a dose (block 328) of the medicinal formulation from the reservoir through the microneedles to the skin or not deliver a dose (block 332) of the medicinal formulation. Upon completion of the delivery of the dose (or if no dose is delivered), the delivery engine 244 may instruct the actuator 208 to remain activated (e.g., the microneedle array remains in position on the skin) (block 336) or to retract (block 340) the microneedle array 200 (or activate the shield 420) from the skin and move to a storage location with the wearable device 204. If the array 200 is retracted (or the shield 420 is activated), the process may be repeated from block 308 where the array is reactivated to puncture the skin of the user. If the array 200 is not retracted (or the shield 420 is not activated), the process may be repeated from block 316 where a subsequent fluid sample is collected thereby performing the diagnostic process a subsequent time.

In the delivery process, there may be no diagnostic process that occurs prior to delivery of a dose of the medicinal formulation. In the standard delivery process, the electronic processor 216 provides an instruction to the delivery engine 244 to deliver a dose (block 344) of the medicinal formulation from the reservoir via the microneedles to the skin. In this standard delivery process, the medicinal formulation can be delivered on a periodic basis to the user, such as on, a daily basis, a twice daily basis, a monthly basis, and the like. Upon completion of the delivery of the dose, the delivery engine 244 may instruct the actuator 208 to remain activated (e.g., remain in position on the skin) (block 348) or to retract (block 352) from the skin and move to a storage location with the wearable device 204. If the array 200 is retracted (or the shield 420 is activated), the process may be repeated from block 308 where the array 200 or shield 420 is reactivated to puncture the skin of the user. If the array 200 is not retracted (or the shield 420 is not activated), the process may be repeated from block 320 where a subsequent dose is delivered to the user.

It should be noted that a plurality of hardware and software based devices, as well as a plurality of different structural components may be utilized to implement the invention. In some embodiments, the invention provides a software application that is executable on a personal or wearable device, such as a smart watch, a ring, a bracelet, and the like. It will be appreciated that some embodiments may be comprised of one or more generic or specialized electronic processors (or “processing devices”) such as microprocessors, digital signal processors, customized processors and field programmable gate arrays (FPGAs) and unique stored program instructions (including both software and firmware) that control the one or more electronic processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment may be implemented as a computer-readable storage medium having computer readable code stored thereon for programming a computer (for example, comprising an electronic processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

Various features and advantages of the invention are set forth in the following claims.

What is claimed is:

1. A wearable device comprising:
a base coupled to a user;
a recessed portion formed in the base;
a microneedle array supported by the base wherein the microneedle array includes a plurality of microneedles; and
an actuator coupled to the microneedle array to move the microneedle array into and out of the recessed portion in the base.

2. The wearable device of claim 1, wherein the actuator comprises one or more springs and a motor.

3. The wearable device of claim 1, wherein the actuator comprises a solenoid.

4. The wearable device of claim 1, further comprising an electronic processor communicatively coupled to the actuator and configured to:
activate the actuator to engage the microneedle array to contact the user,
analyze a fluid sample collected from the user, and
compare data from the fluid sample to a threshold.

5. The wearable device of claim 4, wherein the electronic processor is further configured to:
generate an instruction to deliver a medicinal formulation from a reservoir to the user via the microneedles in the microneedle array.

6. The wearable device of claim 4, wherein the electronic processor is further configured to:
generate an instruction to retract the actuator to remove the microneedles from the user.

7. The wearable device of claim 1, further comprising a reservoir supported by the base, the reservoir containing a medicinal formulation.

8. The wearable device of claim 1, further comprising a port supported by the base, wherein the port is removeably coupleable to a reservoir containing a medicinal formulation.

9. The wearable device of claim 1, wherein the plurality of microneedles in the microneedle array are hollow and in fluid communication with a reservoir containing a medicinal formulation.

10. The wearable device of claim 1, further comprising an electronic processor communicatively coupled to the actuator and configured to:
activate the actuator to engage the microneedle array to contact the user.

11. The wearable device of claim 10, wherein the electronic processor is further configured to:
generate an instruction to deliver a medicinal formulation from a reservoir to the user via the microneedles in the microneedle array.

12. The wearable device of claim 1, wherein the microneedle array is removably coupleable to the actuator.

13. The wearable device of claim 1, wherein the base includes an interactive user interface for inputting and outputting data regarding the user.

14. The wearable device of claim 1, further comprising an electronic processor, and wherein the base includes an interactive user interface for inputting instructions to the electronic processor for activation of the actuator.

15. A wearable device comprising:
a base coupled to a user;
a housing connected to the base;
a microneedle array supported by the housing wherein the microneedle array includes a plurality of microneedles; and
an actuator coupled to a shield in the housing to move the shield and expose the microneedle array to puncture the user's skin.

16. The wearable device of claim 15, wherein the actuator comprises one or more springs and a motor.

17. A method of analyzing a health status of a user, the method comprising:
activating a microneedle array in a wearable device to puncture the user's skin, the microneedle array including a plurality of hollow microneedles;
collecting a fluid sample from the user via the hollow microneedles;
analyzing the fluid sample to generate data of the user;
transmitting the data to a remote electronic processor for review and diagnosis; and
delivering a medicinal formulation from a reservoir in the wearable device to the user via the hollow microneedles based on the diagnosis.

18. The method of claim 17, further comprising retracting the microneedle array from the user's skin.

19. The method of claim 17, further comprising comparing the data to a threshold.

20. The method of claim 17, further comprising replacing the microneedle array in the wearable device.
